(19) United States
(12) Patent Application Publication
(10) Pub. No.: US 2024/0052298 A1
(43) Pub. Date: Feb. 15, 2024

(54) PREPARATION METHOD OF MOFs-COATED CARDIOMYOCYTE CORE-SHELL STRUCTURE

(71) Applicant: Shenzhen Polytechnic, Shenzhen (CN)

(72) Inventors: Xiaolin Li, Shenzhen (CN); Wei Chen, Shenzhen (CN); Liang Zhang, Shenzhen (CN); Tao Gong, Shenzhen (CN)

(21) Appl. No.: 18/260,710

(22) PCT Filed: Apr. 11, 2022
(86) PCT No.: PCT/CN2022/085998

§ 371 Date: Jul. 7, 2023

(30) Foreign Application Priority Data
Aug. 18, 2021 (CN) 202110948712.6

(51) Int. Cl. C12N 5/00; C12N 5/077; B01J 13/04

(52) U.S. Cl. CPC C12N 5/0012; C12N 5/0657; B01J 13/04; C12N 2533/30

(57) ABSTRACT

The present disclosure provides a preparation method of an MOFs-coated cardiomyocyte core-shell structure, and relates to a method for coating cardiomyocytes. The present disclosure aims to solve problems that bio-hybrid microrobots in the prior art lack physical protection and nutrient supply channels and have short life cycles. The method includes the steps of: 1) cleaning; 2) adding trypsin in PBS; 3) digesting; 4) centrifuging; 5) culturing; 6) passaging; 7) preparing a Zn(NO₃)₂·6H₂O aqueous solution and a 2-methylimidazole aqueous solution; and 8) adding the Zn(NO₃)₂·6H₂O aqueous solution and the 2-methylimidazole aqueous solution for culture to obtain the MOFs-coated cardiomyocyte core-shell structure. After 7 days of culture under the same culture conditions, cardiomyocytes having an MOFs shell layer have significantly higher cell density than the cardiomyocytes. An MOFs-coated cardiomyocyte core-shell structure can be obtained according to the present disclosure.

### CROSS REFERENCE TO RELATED APPLICATION

This patent application is a national stage application of International Patent Application No. PCT/CN2022/085998, filed on Apr. 11, 2022, which claims the benefit and priority of Chinese Patent Application No. 202110948712.6 filed with the China National Intellectual Property Administration on Aug. 18, 2021 and entitled with “PREPARATION METHOD OF MOFs-COATED CARDIOMYOCYTE CORE-SHELL STRUCTURE”, the disclosure of which is incorporated by reference herein in its entirety as part of the present application.

### TECHNICAL FIELD

The present disclosure relates to a method for coating cardiomyocytes.

### BACKGROUND

Cardiomyocytes can produce spontaneous and rhythmic contraction without any external assistance. Therefore, it is relatively easy to use cardiomyocytes to make bio-hybrid microrobots. Considering that a single cardiomyocyte is about 100 μm in size and is characterized by spontaneous contraction, a single cardiomyocyte can be used as a driver for hundred-micron-sized microrobots. This will provide a new idea for solving the problems of energy and nutrient supply of microrobots in special working environments (such as the human body).

However, the living bio-driven materials in bio-hybrid microrobots often have short life cycles and poor environmental robustness, and lack appropriate coupling mechanisms between artificial micro-nano structures. This has always been a bottleneck restricting the use of bio-hybrid microrobots in biomedicine. This is mainly due to the lack of necessary nutrient supply and physical protection, as well as effective preparation methods and theories. Most of the existing preparation processes are to first prepare a non-biological robot body structure having a specific structure through physical and chemical methods, and then use specific affinity to attach biological materials to the surface of non-biological materials. The research scale is limited by the size of biological and non-biological materials, which is not conducive to understanding and guiding the optimal design of materials at the molecular level.

### SUMMARY

The present disclosure aims to solve the problems that bio-hybrid microrobots in the prior art lack physical protection and nutrient supply channels and have short life cycles, and to provide a preparation method of a metal-organic frameworks (MOFs)-coated cardiomyocyte core-shell structure.

A preparation method of an MOFs-coated cardiomyocyte core-shell structure is provided, including the following steps:
step 1, discarding a supernatant of an HL-1 mouse cardiomyocyte culture medium, and cleaning HL-1 mouse cardiomyocytes with phosphate buffered saline (PBS) or normal saline;
step 2, adding cleaned HL-1 mouse cardiomyocytes into a cell culture flask or a Petri dish, and adding 0.25% (w/w) trypsin in PBS, so that the 0.25% (w/w) trypsin in PBS is covered over the HL-1 mouse cardiomyocytes;
step 3, putting the cell culture flask or the Petri dish in step 2 into a 37° C. CO₂ incubator for digestion, and adding a culture medium to terminate the digestion to obtain a cell suspension;
step 4, subjecting the cell suspension to centrifugation, and discarding a supernatant to obtain cell pellets;
step 5, resuspending the cell pellets in a fresh culture medium, adding the cell pellets into a cell culture flask or a Petri dish, and culturing the cell culture flask or the Petri dish in the 37° C. CO₂ incubator for 1-7 days to obtain suspension cells;
step 6, subjecting the suspension cells obtained in step 5 to centrifugation, and collecting cell pellets; resuspending the cell pellets in a fresh culture medium, and transferring the cell pellets to a new cell culture flask or a Petri dish for passage, where the passage is conducted in a ratio of 1:(2-8);
step 7, preparing a Zn(NO₃)₂·6H₂O aqueous solution and a 2-methylimidazole aqueous solution, where
the Zn(NO₃)₂·6H₂O aqueous solution in step 7 has a concentration of 1-10 g/L; and
the 2-methylimidazole aqueous solution in step 7 has concentration of 5-20 g/L; and
step 8, separately adding the Zn(NO₃)₂·6H₂O aqueous solution and the 2-methylimidazole aqueous solution to the cell culture flask or the Petri dish obtained in step 6, and transferring the cell culture flask or the Petri dish to the 37° C. CO₂ incubator for culture for 1-7 days to obtain the MOFs-coated cardiomyocyte core-shell structure.

The present disclosure has the following principle:

In order to overcome the inherent difficulties of bio-hybrid microrobots lacking physical protection, nutrient supply channels and optimal design methods, the present disclosure prepares a long-life bio-hybrid microrobot. The present disclosure aims to in situ construct an MOFs physical protection layer on the surface of a biological material by means of biomimetic mineralization. The life support of the biological material is realized by selectively penetrating nutrients and gases necessary for the survival of the biological material.

The present disclosure has the following advantages: The MOFs physical protection layer can selectively penetrate the nutrients and gases necessary for the survival of the biological material, thereby avoiding the influence of cytotoxic substances in the environment on cell lifespan and effectively prolonging the lifespan of cardiomyocytes. After 7 days of culture under the same culture conditions, cardiomyocytes having an MOFs shell layer have significantly higher cell density than the cardiomyocytes.

An MOFs-coated cardiomyocyte core-shell structure can be obtained according to the present disclosure.

The present summary is provided only by way of example and not limitation. Other aspects of the present invention will be appreciated in view of the entirety of the present disclosure, including the entire text, claims, and accompanying figures.

### BRIEF DESCRIPTION OF THE DRAWINGS

FIG. 1 shows micrographs at 100× magnification, with added annotations, where a and b are micrographs of cardiomyocytes in different directions after passage in step 6 of Example 1, and c and d are micrographs of an MOFs-coated cardiomyocyte core-shell structure obtained in step 8 of Example 1; and

FIG. 2 shows micrographs of cardiomyocytes, with added annotations, where a is a micrograph at 40× magnification of 1-IL-1 mouse cardiomyocytes after 7 days of culture, and b is a micrograph at 40× magnification of an MOFs-coated cardiomyocyte core-shell structure obtained in step 8 of Example 1 after 7 days of culture.

### DETAILED DESCRIPTION OF THE EMBODIMENTS

The content of the present disclosure will be further described below in conjunction with the examples, but they should not be construed as limiting the present disclosure. All modifications or substitutions made to methods, steps or conditions of the present disclosure without departing from the essence of the present disclosure fall within the scope of the present disclosure.

Embodiment 1: In this embodiment, a preparation method of an MOFs-coated cardiomyocyte core-shell structure is provided, including the following steps:
step 1, discarding a supernatant of an HL-1 mouse cardiomyocyte culture medium, and cleaning HL-1 mouse cardiomyocytes with PBS or normal saline;
step 2, adding cleaned HL-1 mouse cardiomyocytes into a cell culture flask or a Petri dish, and adding 0.25% (w/w) trypsin in PBS, so that the 025% (w/w) trypsin in PBS is covered over the HL-1 mouse cardiomyocytes;
step 3, putting the cell culture flask or the Petri dish in step 2 into a 37° C. CO₂ incubator for digestion, and adding a culture medium to terminate the digestion to obtain a cell suspension;
step 4, subjecting the cell suspension to centrifugation, and discarding a supernatant to obtain cell pellets;
step 5, resuspending the cell pellets in a fresh culture medium, adding the cell pellets into a cell culture flask or a Petri dish, and culturing the cell culture flask or the Petri dish in the 37° C. CO₂ incubator for 1-7 days to obtain suspension cells;
step 6, subjecting the suspension cells obtained in step 5 to centrifugation, and collecting cell pellets; resuspending the cell pellets in a fresh culture medium, and transferring the cell pellets to a new cell culture flask or a Petri dish for passage, where the passage is conducted in a ratio of 1:(2-8);
step 7, preparing a Zn(NO₃)₂·6H₂O aqueous solution and a 2-methylimidazole aqueous solution, where
the Zn(NO₃)₂·6H₂O aqueous solution in step 7 has a concentration of 1-10 g/L; and
the 2-methylimidazole aqueous solution in step 7 has concentration of 5-20 g/L; and
step 8, separately adding the Zn(NO₃)₂·6H₂O aqueous solution and the 2-methylimidazole aqueous solution to the cell culture flask or the Petri dish obtained in step 6, and transferring the cell culture flask or the Petri dish to the 37° C. CO₂ incubator for culture for 1-7 days to obtain the MOFs-coated cardiomyocyte core-shell structure.

The HL-1 mouse cardiomyocyte culture medium in step 1 of this embodiment is purchased from Hunan Fenghui Biotechnology Co., Ltd.

Embodiment 2: A difference between this embodiment and Embodiment 1 is that: the cleaning in step 1 is conducted once or twice. Other steps are the same as those in Embodiment 1.

Embodiment 3: Differences between this embodiment and any one of Embodiment 1 and 2 are that: a volume fraction of CO₂ in the CO₂ incubator in step 3 is 5%; the culture medium in step 3 is composed of 90% (w/w) Dulbecco's Modified Eagle Medium (DMEM) High Glucose, 9% (w/w) fetal bovine serum (FBS) and 1% (w/w) double antibody; and the double antibody has a concentration of 100 U/mL. Other steps are the same as those in Embodiment 1 or 2.

Embodiment 4: A difference between this embodiment and any one of Embodiments 1 to 3 is that the culture medium in step 3 and the HL-1 mouse cardiomyocytes in the cell culture flask or the Petri dish have a volume ratio of (6-8):1. Other steps are the same as those in Embodiments 1 to 3.

Embodiment 5: A difference between this embodiment and any one of Embodiments 1 to 4 is that the digestion in step 3 is conducted for 0.5-2 min. Other steps are the same as those in Embodiments 1 to 4.

Embodiment 6: A difference between this embodiment and any one of Embodiments 1 to 5 is that the centrifugation in step 4 is conducted at 500-2,000 r/min for 3-6 min. Other steps are the same as those in Embodiments 1 to 5.

Embodiment 7: Differences between this embodiment and any one of Embodiments 1 to 6 are that: a volume fraction of CO₂ in the CO₂ incubator in step 5 is 5%; the fresh culture medium in step 5 is composed of 90% (w/w) DMEM High Glucose, 9% (w/w) FBS and 1% (w/w) double antibody; and the double antibody has a concentration of 100 U/mL. Other steps are the same as those in Embodiments 1 to 6.

Embodiment 8: Differences between this embodiment and any one of Embodiments 1 to 7 are that: the fresh culture medium in step 6 is composed of 90% (w/w) DMEM High Glucose, 9% (w/w) FBS and 1% (w/w) double antibody; the double antibody has a concentration of 100 U/mL; and the centrifugation in step 6 is conducted at 500-2,000 r/min for 3-6 min. Other steps are the same as those in Embodiments 1 to 7.

Embodiment 9: Differences between this embodiment and any one of Embodiments 1 to 8 are that: a volume fraction of CO₂ in the CO₂ incubator in step 8 is 5%; and the Zn(NO₃)₂·6H₂O aqueous solution and the culture medium in step 8 have a volume ratio of 6.25×10⁻⁵ to 1.67×10⁻³. Other steps are the same as those in Embodiments 1 to 8.

Embodiment 10: A difference between this embodiment and any one of Embodiments 1 to 9 is that the 2-methylimidazole aqueous solution and the culture medium in step 8 have a volume ratio of 6.25×10⁻⁵ to 1.67×10⁻³. Other steps are the same as those in Embodiments 1 to 9.

The present disclosure will be described in detail below with reference to the drawings and examples.

Example 1: A preparation method of an MOFs-coated cardiomyocyte core-shell structure was provided, including the following steps:
step 1, a supernatant of an HL-1 mouse cardiomyocyte culture medium was discarded, and HL-1 mouse cardiomyocytes were cleaned with PBS once;
the HL-1 mouse cardiomyocyte culture medium in step 1 was purchased from Hunan Fenghui Biotechnology Co., Ltd.;
step 2, 1 mL of cleaned HL-1 mouse cardiomyocytes were added into a T25 cell culture flask, and 1 mL of 0.25% (w/w) trypsin in PBS was added, so that the 0.25% (w/w) trypsin in PBS was covered over the HL-1 mouse cardiomyocytes;
step 3, the cell culture flask in step 2 was put into a 37° C. CO₂ incubator for digestion for 0.5 min, and 6 mL of culture medium was added to terminate the digestion to obtain a cell suspension:
the volume fraction of CO₂ in the CO₂ incubator in step 3 was 5%;
the culture medium in step 3 was composed of 90% (w/w) DMEM High Glucose, 9% (w/w) FBS and 1% (w/w) double antibody; and the double antibody had a concentration of 100 U/mL;
step 4, the cell suspension was subjected to centrifugation, and a supernatant was discarded to obtain cell pellets;
the centrifugation in step 4 was conducted at 1,000 r/min for 4 min;
step 5, the cell pellets were resuspended in 6 mL of fresh culture medium and added into a T25 cell culture flask, and the cell culture flask was cultured in the 37° C. CO₂ incubator for 2 days;
the volume fraction of CO₂ in the CO₂ incubator in step 5 was 5%;
the fresh culture medium in step 5 was composed of 90% (w/w) DMEM High Glucose, 9% (w/w) FBS and 1% (w/w) double antibody; and the double antibody had a concentration of 100 U/mL;
step 6, the suspension cells obtained in step 5 were subjected to centrifugation, and cell pellets were collected; the cell pellets were resuspended in 1 mL of fresh culture medium, and transferred to a new T25 cell culture flask for passage, where the passage was conducted in a ratio of 1:2;
the fresh culture medium in step 6 was composed of 90% (w/w) DMEM High Glucose, 9% (w/w) FBS and 1% (w/w) double antibody; and the double antibody had a concentration of 100 U/mL;
the centrifugation in step 6 was conducted at 1,000 r/min for 4 min;
step 7, a Zn(NO₃)₂·6H₂O aqueous solution and a 2-methylimidazole aqueous solution were prepared;
the Zn(NO₃)₂·6H₂O aqueous solution in step 7 had a concentration of 5 g/L; and
the 2-methylimidazole aqueous solution in step 7 had concentration of 10 g/L; and
step 8, 1 μL of Zn(NO₃)₂·6H₂O aqueous solution and 1 μL of 2-methylimidazole aqueous solution were separately added to the cell culture flask obtained in step 6, and the cell culture flask was transferred to the 37° C. CO₂ incubator for culture for 2 days to obtain the MOPs-coated cardiomyocyte core-shell structure; and
the volume fraction of CO₂ in the CO₂ incubator in step 8 was 5%.

Example 2: A preparation method of an MOFs-coated cardiomyocyte core-shell structure was provided, including the following steps:
step 1, a supernatant of an HL-1 mouse cardiomyocyte culture medium was discarded, and HL-1 mouse cardiomyocytes were cleaned with PBS once;
the HL-1 mouse cardiomyocyte culture medium in step 1 was purchased from Hunan Fenghui Biotechnology Co., Ltd.;
step 2, 1 mL of cleaned HL-1 mouse cardiomyocytes were added into a T25 cell culture flask, and 0.5 ml, of 0.25% (w/w) trypsin in PBS was added, so that the 0.25% (w/w) trypsin in PBS was covered over the HL-1 mouse cardiomyocytes;
step 3, the cell culture flask in step 2 was put into a 37° C. CO₂ incubator for digestion for 1 min, and 6 mL of culture medium was added to terminate the digestion to obtain a cell suspension;
the volume fraction of CO₂ in the CO₂ incubator in step 3 was 5%;
the culture medium in step 3 was composed of 90% (w/w) DMEM High Glucose, 9% (w/w) FBS and 1% (w/w) double antibody; and the double antibody had a concentration of 100 U/mL;
step 4, the cell suspension was subjected to centrifugation, and a supernatant was discarded to obtain cell pellets;
the centrifugation in step 4 was conducted at 2,000 r/min for 4 min;
step 5, the cell pellets were resuspended in 8 mL of fresh culture medium and added into a T25 cell culture flask, and the cell culture flask was cultured in the 37° C. CO₂ incubator for 2 days;
the volume fraction of CO₂ in the CO₂ incubator in step 5 was 5%;
the fresh culture medium in step 5 was composed of 90% (w/w) DMEM High Glucose, 9% (w/w) FBS and 1% (w/w) double antibody; and the double antibody had a concentration of 100 U/mL;
step 6, the suspension cells obtained in step 5 were subjected to centrifugation, and cell pellets were collected; the cell pellets were resuspended in 1 mL of fresh culture medium, and transferred to a new T25 cell culture flask for passage, where the passage was conducted in a ratio of 1:4;
the fresh culture medium in step 6 was composed of 90% (w/w) DMEM High Glucose, 9% (w/w) FBS and 1% (w/w) double antibody; and the double antibody had a concentration of 100 U/mL;
the centrifugation in step 6 was conducted at 2,000 r/min for 4 min;
step 7, a Zn(NO₃)₂·6H₂O aqueous solution and a 2-methylimidazole aqueous solution were prepared;
the Zn(NO₃)₂·6H₂O aqueous solution in step 7 had a concentration of 10 g/L; and
the 2-methylimidazole aqueous solution in step 7 had concentration of 20 g/L; and
step 8, 5 μL of Zn(NO₃)₂·6H₂O aqueous solution and 5 μL of 2-methylindazole aqueous solution were separately added to the cell culture flask obtained in step 6, and the cell culture flask was transferred to the 37° C. CO₂ incubator for culture for 2 days to obtain the MOFs-coated cardiomyocyte core-shell structure; and
the volume fraction of CO₂ in the CO₂ incubator in step 8 was 5%.

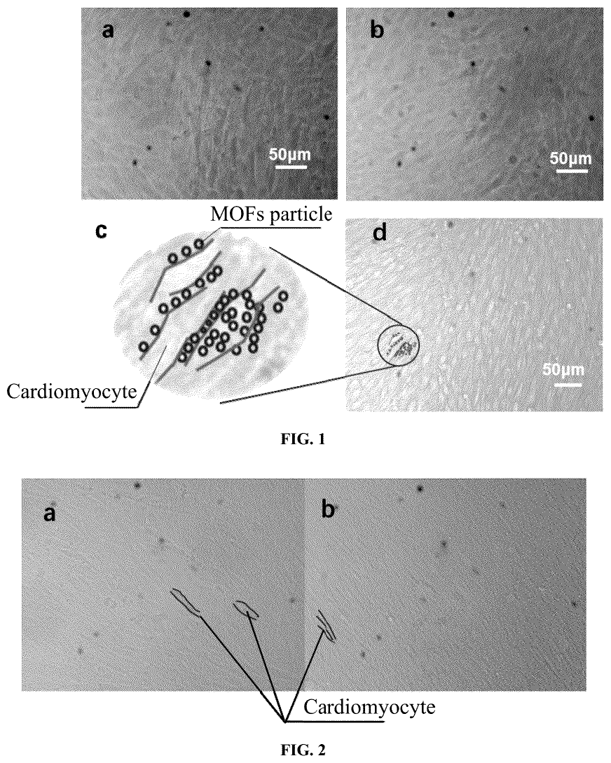

FIG. 1 shows micrographs at 100× magnification, where a and b are micrographs of cardiomyocytes in different directions after passage in step 6 of Example 1, and c and d are micrographs of an MOFs-coated cardiomyocyte core-shell structure obtained in step 8 of Example 1.

As can be seen from FIG. 1, it is found through the in vitro culture of cardiomyocytes (a and b) that the cardiomyocyte thickness decreases with the progress of the culture process, while the spreading area gradually increases, and membrane potential conduction occurs between either end of the cardiomyocyte and the surrounding cardiomyocyte. It can be seen that the cardiomyocytes can also perform a complete rhythmic contraction in an in Vitro culture medium environment. c and d are micrographs of cardiomyocytes after co-culture of cardiomyocytes with MOFs precursors (metal ions and ligands) for 2 days. Compared with a and b, d obviously shows an increase in crystal particles. Because the MOFs precursor solution is soluble in water, it indicates that under the culture conditions of cardiomyocytes, MOFs precursors can form MOFs particles on the surface of the cardiomyocyte membrane without affecting the morphology and bioactivity of the cells. This study shows that the mineralized synthesis of MOFs particles is feasible.

FIG. 2 shows micrographs of cardiomyocytes, where a is a micrograph at 40× magnification of HL-1 mouse cardiomyocytes after 7 days of culture, and b is a micrograph at 40× magnification of the MOFs-coated cardiomyocyte core-shell structure obtained in step 8 of Example 1.

As can be seen from FIG. 2, after culturing each of the passaged cardiomyocytes obtained from the same batch of cells for 7 days, the cell density of MOFs-coated cardiomyocytes is significantly higher than that of non-coated cardiomyocytes, indicating that the MOFs shell layer is beneficial to the growth of cardiomyocytes. In the same culture time, the survival of cells can be guaranteed to the utmost extent.

The above examples only express several embodiments of the present application, and the description thereof is more specific and detailed, but cannot be construed as a limitation on the scope of the present disclosure. It should be noted that, for a person of ordinary skill in the art, several variations and improvements can be made without departing from the concept of the present application, all of which fall within the protection scope of the present application. Therefore, the protection scope of this application shall be subject to the appended claims.

1. A preparation method of a metal-organic frameworks (MOFs)-coated cardiomyocyte core-shell structure, comprising the following steps:
adding a Zn(NO₃)₂·6H₂O aqueous solution and a 2-methylimidazole aqueous solution into an HL-1 mouse cardiomyocyte for passage, and culturing the cardiomyocyte in a 37° C. CO₂ incubator for 1-7 days to obtain the MOFs-coated cardiomyocyte core-shell structure; wherein
the passage is conducted in a ratio of 1:(2-8);
the Zn(NO₃)₂·6H₂O aqueous solution has a concentration of 1-10 g/L; and
the 2-methylimidazole aqueous solution has a concentration of 5-20 g/L.

2. A preparation method of an MOFs-coated cardiomyocyte core-shell structure, comprising the following steps:
step 1, discarding a supernatant of an HL-1 mouse cardiomyocyte culture medium, and cleaning HL-1 mouse cardiomyocytes with phosphate buffered saline (PBS) or normal saline;
step 2, adding cleaned HL-1 mouse cardiomyocytes into a cell culture flask or a Petri dish, and adding 0.25% (w/w) trypsin in PBS, so that the 0.25% (w/w) trypsin in PBS is covered over the HL-1 mouse cardiomyocytes;
step 3, putting the cell culture flask or the Petri dish in step 2 into a 37° C. CO₂ incubator for digestion, and adding a culture medium to terminate the digestion to obtain a cell suspension;
step 4, subjecting the cell suspension to centrifugation, and discarding a supernatant to obtain cell pellets;
step 5, resuspending the cell pellets in a fresh culture medium, adding the cell pellets into a cell culture flask or a Petri dish, and culturing the cell culture flask or the Petri dish in the 37° C. CO₂ incubator for 1-7 days to obtain suspension cells;
step 6, subjecting the suspension cells obtained in step 5 to centrifugation, and collecting cell pellets; resuspending the cell pellets in a fresh culture medium, and transferring the cell pellets to a new cell culture flask or a Petri dish for passage, wherein the passage is conducted in a ratio of 1:(2-8);
step 7, preparing a Zn(NO₃)₂·6H₂O aqueous solution and a 2-methylimidazole aqueous solution, wherein
the Zn(NO₃)₂·6H₂O aqueous solution has a concentration of 1-10 g/L; and
the 2-methylimidazole aqueous solution has concentration of 5-20 g/L; and
step 8, separately adding the Zn(NO₃)₂·6H₂O aqueous solution and the 2-methylimidazole aqueous solution to the cell culture flask or the Petri dish obtained in step 6, and transferring the cell culture flask or the Petri dish to the 37° C. CO₂ incubator for culture for 1-7 days to obtain the MOFs-coated cardiomyocyte core-shell structure.

3. The preparation method of an MOFs-coated cardiomyocyte core-shell structure according to claim 2, wherein the cleaning in step 1 is conducted once or twice.

4. The preparation method of an MOFs-coated cardiomyocyte core-shell structure according to claim 2, wherein a volume fraction of CO₂ in the CO₂ incubator in step 3 is 5%.

5. The preparation method of an MOFs-coated cardiomyocyte core-shell structure according to claim 2, wherein the culture medium in step 3 comprises 90% (w/w) Dulbecco's Modified Eagle Medium (DMEM) High Glucose, 9% (w/w) fetal bovine serum (FBS) and 1% (w/w) double antibody; and the double antibody has a concentration of 100 U/mL.

6. The preparation method of an MOFs-coated cardiomyocyte core-shell structure according to claim 2, wherein the culture medium in step 3 and the HL-1 mouse cardiomyocytes in the cell culture flask or the Petri dish have a volume ratio of (6-8):1.

7. The preparation method of an MOFs-coated cardiomyocyte core-shell structure according to claim 2, wherein the digestion in step 3 is conducted for 0.5-2 min.

8. The preparation method of an MOFs-coated cardiomyocyte core-shell structure according to claim 2, wherein the centrifugation in step 4 is conducted at 500-2,000 r/min for 3-6 min.

9. The preparation method of an MOFs-coated cardiomyocyte core-shell structure according to claim 2, wherein a volume fraction of CO₂ in the CO₂ incubator in step 5 is 5%.

10. The preparation method of an MOFs-coated cardiomyocyte core-shell structure according to claim 2, wherein the fresh culture medium in step 5 comprises 90% (w/w) DMEM High Glucose, 9% (w/w) FBS and 1% (w/w) double antibody; and the double antibody has a concentration of 100 U/mL.

11. The preparation method of an MOFs-coated cardiomyocyte core-shell structure according to claim 2, wherein the fresh culture medium in step 6 comprises 90% (w/w) DMEM High Glucose, 9% (w/w) FBS and 1% (w/w) double antibody; and the double antibody has a concentration of 100 U/mL.

12. The preparation method of an MOFs-coated cardiomyocyte core-shell structure according to claim 2, wherein the centrifugation in step 6 is conducted at 500-2,000 r/min for 3-6 min.

13. The preparation method of an MOFs-coated cardiomyocyte core-shell structure according to claim 2, wherein a volume fraction of CO₂ in the CO₂ incubator in step 8 is 5%; and the Zn(NO₃)₂·6H₂O aqueous solution and the culture medium in step 8 have a volume ratio of 6.25×10⁻⁵ to 1.67×10⁻³.

14. The preparation method of an MOFs-coated cardiomyocyte core-shell structure according to claim 2, wherein the 2-methylimidazole aqueous solution and the culture medium in step 8 have a volume ratio of 6.25×10⁻⁵ to 1.67×10⁻³.

15. An MOFs-coated cardiomyocyte core-shell structure prepared by the preparation method according to claim 1, comprising cardiomyocytes and an MOFs physical protection layer for coating the cardiomyocytes.

16. (canceled)

17. The preparation method of an MOFs-coated cardiomyocyte core-shell structure according to claim 4, wherein the culture medium in step 3 comprises 90% (w/w) Dulbecco's Modified Eagle Medium (DMEM) High Glucose, 9% (w/w) fetal bovine serum (FBS) and 1% (w/w) double antibody; and the double antibody has a concentration of 100 U/mL.

18. The preparation method of an MOFs-coated cardiomyocyte core-shell structure according to claim 9, wherein the fresh culture medium in step 5 comprises 90% (w/w) DMEM High Glucose, 9% (w/w) FBS and 1% (w/w) double antibody; and the double antibody has a concentration of 100 U/mL.

19. The preparation method of an MOFs-coated cardiomyocyte core-shell structure according to claim 11, wherein the centrifugation in step 6 is conducted at 500-2,000 r/min for 3-6 min.
